(19) United States
(12) Patent Application Publication
(10) Pub. No.: US 2004/0204468 A1
(43) Pub. Date: Oct. 14, 2004

(54) Pharmaceutical preparation for a topical treatment of fever blisters/cold sores, and minor lip irritations

(76) Inventor: Charles Martin Levin, Hillsborough, CA (US)

Correspondence Address: Charles M. Levin D.D.S., 1340 Brandt Rd., Hillsborough, CA, 94010, US

(21) Appl. No.: 10/408,880

(22) Filed: Apr. 9, 2003

(51) Int. Cl. A61K 31/4164; A61K 31/355; A61K 31/24

(52) U.S. Cl. 514/389; 514/458; 514/537

(57) ABSTRACT

This invention was created with a unique and specific Formulation and a specific Method for the treatment of fever blister/cold sores, and minor lip irritations. The specific Formulation consists of a topical gel solution. The specific Method of treatment is to apply the gel directly on the lesion every four hours. The formulations significant ingredients are: lidocaine hcl 1.00%, tocopheryl acetate 0.2%, and allantoin 0.1%. The formulation and the method were clinically tested. The study demonstrated a marked reduction of pain and a reduction in healing time as compared to existing formulations and methods.

### TECHNICAL

This invention relates to an exact Formulation of a clear odorless liquid gel for a topical Method of treatment of fever blisters/cold sores, and minor lip irritations.

### BACKGROUND OF THE INVENTION

Fever blisters/cold sores and minor lip irritations have long been an uncomfortable and unsightly problem. Untreated their duration can be ten days to two weeks. These lesions are classified as: 1. Herpes Simplex type 1 ( HSV1 ). HSV1 occurs of the lips and mucous membrane and is very contagious. Once you have been infected with this virus it stays in your body forever. The most common causes for an outbreak are cold or fever, stress, exposure to extreme temperature, and sunlight. The symptoms are a very painful dull constant pain. 2. Minor lip irritations. Most irritations appear on the mucous membrane of the lips as small ulcerations called Apthus Ulcers or as extreme dry cracking or braking of the lip surface epithelium. These lesions are not contagious. The most common cause is irritation, allergic response, hot and spicy foods, exposure to sunlight, wind, extreme temperatures and virus. The symptoms are painful dull constant pain and very sensitive to local stimulation such as hot, spicy, sour foods or touching. It is estimated that about forty million Americas suffer from Cold Sores/Fever Blisters and Minor Lip Irritations. Most remedies only offer temporary relief, and have little effect of healing. Lidocaine HCL, Vitamin E acetate and Allantoin in combination with a formula proved to be a new and effective treatment. Lidocaine HCL is solution with high viscosity and low surface tension—thus it spreads well and adheares to the oral mucous membrane. The topical application is absorbed into the mucous membrane and subcutaneous layer and effects the neuronal membrane and thereby prevents initiation and transmission of nerve impulse. Lidocaine is an effective anti-viral agent when used in combination with pantothenic acid. (Haines, U.S. Pat. No. 4,628,063). Lidocain is used as a topical anesthetic in many dental procedures. It has been shown to temporarily relieve pain through absorption of the mucous membrane or epithelium. Vitamin e acetate and its derivatives are substances that are considered antioxidants and thereby remove free radicals. (Giorgio U.S. Pat. No. 6,280,752) it was proven that vitamin e acetate has the physical properties such as to make it suitable for use as a main component of the lipophilic phase of ointments. (Giorgio pat. #wo 98/10793). Allantoin has been used in several formulations for the promotion of epithelial stimulation. It was used in combination with primary medicinal ingredients, aloe vera, and silicon dioxide in the form of a gel. (Schulman U.S. Pat. No. 5,503,822. he showed that this gel was an effective formulation when used for the treatment of oral mucous membrane ulcerations it reduce healing time by an average of three days. Allantoin has been used in several formulations indicated for use on the epithelium to help heal or prevent minor skin irritations. The invention uses these three significant ingredients, Lidocain HCL, Vitamin E Acetate and Allatoin in combination with other ingredients for a new and effective treatment of lesions of the lip.

### SUMMUARY OF THE INVENTION

This invention provides a new and effective Formulation and Method to treat fever blistes/cold sores, and minor lip irritations. The formulation consists of three primary medicinal ingredients, lindocain hcl, topopheryl acetate, and allantoin. This combination creates a new product that has demonstrated through patient testing to reduce pain and improve healing time. This product is unique and different because it can be applied on the submucousal epithelium tissue layer, the cutaneous epithelium layer and the oral mucous membrane layer of the lips.

### DETAILED DESCRIBTION OF INVENTION

A formulation was created which is a clear white odorless slightly viscous gel, which flows from a plastic container or dropper. It was laboratory tested for stability at room temperatures, for microbiological contamination and analytical method for assay of the active ingredient, lidocain hcl. All tests meet the FDA standards for over the counter products. The formulation consisted of the following:

| Ingredients | percentage wt. |
|---|---|
| 1. lidocain hcl | 1-2 |
| 2. purified water | >90 |
| 3. glycerin | 3-5 |
| 4. sorbitol | 3-5 |
| 5. hydroxyethycellulose | 1-3 |
| 6. peg 40 hydrogenated castor oil | 0.5-2 |
| 7. citrc acid | 0.1-0.75 |
| 8. potassium sorbate | 0.05-0.2 |
| 9. sodium benzoate | 0.05-0.2 |
| 10. sodium citrate | 0.05-0.2 |
| 11. tocopheryl acetate | 0.2-0.5 |
| 12. allantoin | 0.1-0.2 |
| 13. methylparaben | 0.05-0.2 |
| 14. magnesium ascorbyl phosphate | 0.075-0.09 |
| 15. monoammonium glycyrrhizinate | 0.05-0.075 |

Several individuals tested the formulation and reported their findings. Each person was given the following instructions; clean and dry the sore with a cotton wipe, apply the formula directly to the sore with an applicator tip or a clean finger, apply every four (4) to six (6) hours until the sore healed. They were instructed to apply at the first sign of symptoms. Each patient filled out an evaluation form describing the results of the treatment.

### Evaluation Results

1. Twenty-eight year old female had an uncomfortable dry cracking lesion on her lip. She noted after application of the formula a relief of the pain and discomfort and the sore healed in two (2) days.

2. Thirty-five year old female reported several canker sores on the oral mucous membrane of the lips and cheeks with associated constant dull pain. She has had a history of such lesions. She reported relief of pain and resolution of the lesions in five days.

3. Sixty year old male had a reoccurring apthous ulcer of the unattached gingiva that was painful on stimulation. The formula relieved the pain and healed in six (6) days.

4. Sixty year old female described a canker sore causing a dull constant pain. She claims that the formula relieved the pain and the lesion healed in two (2) days.

5. Another individual who did not give the age or sex stated that the formula was helpful and cleared the sore ulcer in two (2) days.

6. Thirty-three year old female has a history of cold sores, which occur, on her lip causing a dull constant tingling pain. She noted pain relief and the lesion healed in one (1) week.

The above examples show the effectiveness of this invention, namely that the Formulation and Method is a unique and an effective treatment modality for lesions of the lips. The vast majority of the individuals tested reported a significant reduced healing time and a reduction of pain as compared to no treatment or compared to other treatments that they have used.

References

1. Burket's Oral Medicine Diagnosis and Treatment Fouth Edition, 95-97, 97-100

2. Thoma and Goldman's Oral Pathology Fifth Edition, 858, 1021

3. Dorland's Medical Dictionary Twenty third Edition

4. Physician's Desk Reference Forty third Edition

1. This formulation of Lidocaine HCL, Tocopheryl Acetate and Allantoin in a product vehicle is effective in the treatment of fever blister/ cold sores, and minor lip irritations.

2. The method of treating fever blisters/cold sores and minor lip irritations is to apply the formula topically directly to the sore after cleaning and drying. The application is applied directly on the sore and is applied every 4 to 6 hr. This method is continued until the sore heals. This method is effective in reducing pain and reducing dealing time.

3. The formulation and method produces reduced healing time. The healing time is significantly reduced. A typical lesion takes ten (10) days to two (2) weeks to heal when no treatment is used.

4. The formulation and method reduces pain associated with these lesions. All patients in the study found significant reduction in pain as a result of topical application and formulation.

5. The formulation and method penetrates the mucous membrane and does not create a film on the surface of the lips or skin. The formulation and method produces a normal healed epithelium and mucous membrane.

6. The formulation and method is non-toxic and does not cause any abnormal side effects.
